(19) United States
(12) Patent Application Publication
(10) Pub. No.: US 2021/0251309 A1
(43) Pub. Date: Aug. 19, 2021

(54) WEARABLE DEVICE AND SYSTEM FOR DETECTING PARAMETERS OF A USER

(71) Applicants: Zhengrong TANG, Sunnyvale, CA (US); Wenli Zheng, Shanghai (CN); Zhen Cai, Shanghai (CN)

(72) Inventors: Zhengrong TANG, Sunnyvale, CA (US); Wenli Zheng, Shanghai (CN); Zhen Cai, Shanghai (CN)

(21) Appl. No.: 16/827,160

(22) Filed: Mar. 23, 2020

(30) Foreign Application Priority Data
Feb. 14, 2020 (CN) PCT/CN2020/075326

(51) Int. Cl. A41D 1/00; A61B 5/0408; A61B 5/00; D02G 3/44

(52) U.S. Cl. CPC A41D 1/002; A61B 5/0408; D10B 2401/18; D02G 3/441; A61B 2562/0219; A61B 5/6804

(57) ABSTRACT

A wearable device and a system, the wearable device including a wearable textile body including a plurality of interwoven or knitted yarns; one or more textile sensors each located at an area on the wearable textile body, each textile sensor including a plurality of functional yarns integrally interwoven or knitted with the yarns of the wearable textile body at the located area; and an interface communicatively coupled with the one or more textile sensors.

This disclosure claims priority to International Application No. PCT/CN2020/075326 filed on Feb. 14, 2020, which is incorporated herein by reference in its entirety.

### BACKGROUND

Wearable devices are designed to be worn on living beings and used for a variety of functions, for example, monitoring the living beings' vital signals or data related to health and fitness, location, or even biofeedback indicating emotions. Such devices have become increasingly common in daily life. Many wearable devices have been developed and evolved to include powerful sensor technologies that can collect and deliver information about their surroundings. Examples of wearable devices include various types of computerized wristwatches, fitness tracking bands, smart glasses, or the like. Some issues related to conventional wearable devices include how users look or feel when wearing them and user-friendliness.

### SUMMARY

In some embodiments, an exemplary wearable device includes a wearable textile body including a plurality of interwoven or knitted yarns; one or more textile sensors each located at an area on the wearable textile body, each textile sensor including a plurality of functional yarns integrally interwoven or knitted with the yarns of the wearable textile body at the located area; and an interface communicatively coupled with the one or more textile sensors.

In some embodiments, an exemplary system includes a wearable device and a control module. The wearable device includes a wearable textile body including a plurality of interwoven or knitted yarns; one or more textile sensors each located at an area on the wearable textile body, each textile sensor including a plurality of functional yarns integrally interwoven or knitted with the yarns of the wearable textile body at the located area; and an interface communicatively coupled with the one or more textile sensors. The control module includes an interface to receive signals from the wearable device; a memory for storing the received signals or data; and a processor coupled with the memory and to process the received signals.

Additional features and advantages of the present disclosure will be set forth in part in the following detailed description, and in part will be obvious from the description, or may be learned by practice of the present disclosure. The features and advantages of the present disclosure will be realized and attained by means of the elements and combinations particularly pointed out in the appended claims.

It is to be understood that the foregoing general description and the following detailed description are exemplary and explanatory only, and are not restrictive of the disclosed embodiments.

### BRIEF DESCRIPTION OF THE DRAWINGS

The accompanying drawings, which comprise a part of this specification, illustrate several embodiments and, together with the description, serve to explain the principles and features of the disclosed embodiments. In the drawings:

FIG. 1 is a schematic diagram of an exemplary wearable T-shirt device, according to some embodiments of the present disclosure.

FIG. 2 is an enlarged diagram of a part of the exemplary wearable T-shirt device shown in FIG. 1, according to some embodiments of the present disclosure.

FIG. 3 is a schematic diagram of another exemplary wearable T-shirt device, according to some embodiments of the present disclosure.

FIG. 4 is a schematic diagram of an exemplary wearable long sleeve T-shirt device, according to some embodiments of the present disclosure.

FIG. 5 is a schematic diagram of an exemplary wearable band device, according to some embodiments of the present disclosure.

FIGS. 6A and 6B are a schematic diagrams of an exemplary wearable sock device, according to some embodiments of the present disclosure.

FIG. 7 is a schematic diagram of an exemplary wearable glove device, according to some embodiments of the present disclosure.

FIG. 8 is a schematic diagram of an exemplary system including a wearable device, according to some embodiments of the present disclosure.

### DETAILED DESCRIPTION

Reference will now be made in detail to exemplary embodiments, examples of which are illustrated in the accompanying drawings. The following description refers to the accompanying drawings in which the same numbers in different drawings represent the same or similar elements unless otherwise represented. The embodiments set forth in the following description of exemplary embodiments do not represent all embodiments consistent with the invention. Instead, they are merely examples of devices, systems and methods consistent with aspects related to the invention as recited in the appended claims.

Conventional wearable devices may be complicated, uncomfortable, and inconvenient for a user (e.g., a human being or other living being) wearing them. Embodiments of the present disclosure provide improvements over conventional wearable devices. For example, some embodiments interweave or knit a textile sensor into a wearable textile item (e.g., a garment, a sock, a glove, or the like), which can improve a wearing experience of the user and detection precision of the sensor.

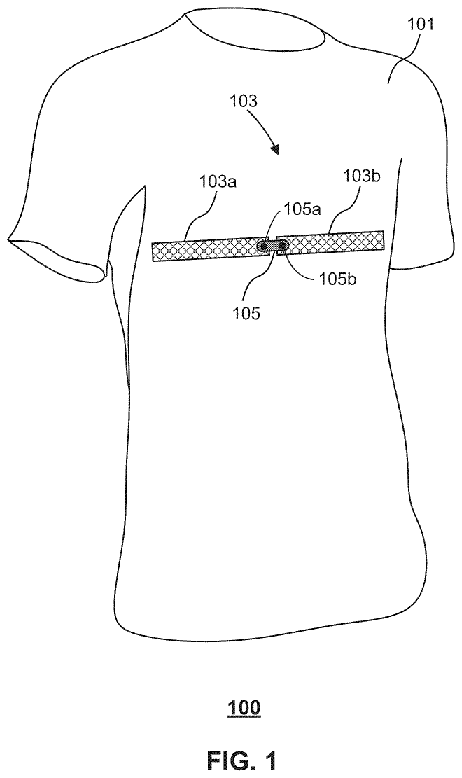

FIG. 1 is a schematic diagram of an exemplary wearable T-shirt device 100, according to some embodiments of the present disclosure. As shown in FIG. 1, wearable T-shirt device 100 includes a wearable textile body 101 and one or more textile sensors, e.g., two textile sensors 103a and 103b, collectively referred to as a textile sensor 103. Although shown as a T-shirt, it is appreciated that wearable textile body 101 can be in the form of other wearable textile items, such as a sweatshirt, underwear, a hat, a wearable band, or the like. Wearable textile body 101 can be formed of a plurality of yarns which are interwoven or knitted to produce a textile form suitable for wear by a user, such as a human being or other living being. The plurality of yarns of wearable textile body 101 can be nonfunctional and not designed to detect parameters of the user. Textile sensor 103 can include a plurality of functional yarns which are integrally interwoven or knitted with the yarns of wearable textile body 101. Integrally interwoven or knitted as used in the present disclosure refers to interweaving or knitting a plurality of yarns into a single piece of cloth. Functional yarn used in the present disclosure refers to a yarn that can be used to detect various parameters of the user, such as physiological parameters, chemical parameters, local pressure, movement, gesture, or the like. For example, at an area where textile sensor 103 is located on wearable textile body 101, functional yarns of textile sensor 103 can be integrally interwoven or knitted with nonfunctional yarns of wearable textile body 101 to form a single piece of wearable cloth.

Functional yarn can include, but is not limited to, conductive yarn, pressure yarn, strain yarn, tension yarn, or the like. Conductive yarn, pressure yarn, strain yarn, or tension yarn can be used to detect electrical signals, pressure signals, strain signals or tension signals, respectively. In some embodiments, the functional yarn can include a plurality of functional fibers twisted or interlaced together. The functional fiber can include, but is not limited to, conductive fiber, pressure fiber, strain fiber, tension fiber, or the like. The conductive fiber can detect and deliver electrical signals, such as physiological electrical signals of the user. The pressure fiber, strain fiber, or tension fiber can detect pressure, strain, or tension applied to the pressure fiber, strain fiber, or tension fiber, respectively. For example, an electrical resistance of the pressure fiber, strain fiber, or tension fiber can change as the pressure, strain, or tension applied to the pressure fiber, strain fiber, or tension fiber changes, respectively. Therefore, the change in electrical resistance of the pressure fiber, strain fiber, or tension fiber can be detected and used to measure a state or change in the pressure, strain, or tension applied to the pressure fiber, strain fiber, or tension fiber, respectively. The functional yarn can include a plurality of conductive fibers, pressure fibers, strain fibers, tension fibers, or a combination thereof.

In some embodiments, textile sensor 103 can include a plurality of the same type of functional yarns. For example, textile sensor 103 can include a plurality of conductive yarns, pressure yarns, strain yarns, or tension yarns to form an electrical textile sensor, a pressure textile sensor, a strain textile sensor, or a tension textile sensor, respectively. Alternatively, textile sensor 103 can include a plurality of different types of yarns to form a combined textile sensor. For example, textile sensor 103 can include a plurality of conductive yarns and pressure yarns to act as a combination of electrical textile sensor and pressure textile sensor.

In some embodiments, one or more textile sensors 103 of wearable T-shirt device 100 can be of the same type, such as electrical textile sensor, pressure textile sensor, strain textile sensor, tension textile sensor, combined sensor, or other suitable type of sensors. Alternatively, in some embodiments, one or more textile sensors 103 of wearable T-shirt device 100 can be of different types. For example, wearable T-shirt device 100 can include one or more electrical textile sensors and one or more tension textile sensors.

In some embodiments, wearable T-shirt device 100 can include an interface 105 that is communicatively coupled with textile sensor 103. Interface 105 can be coupled to an external device, such as a control module, a smart device, or a computer (not shown in FIG. 1), in a wired or wireless manner. For example, as shown in FIG. 1, interface 105 can include a plurality of contacts, e.g., contact 105a and contact 105b, connected to textile sensors 103a and 103b, respectively, for connecting to the external device. As another example, interface 105 can include a wireless transmitter or transceiver which can transmit signals to the external device or receive signals or instructions therefrom. In some embodiments, interface 105 can include a docking station, e.g., a recess, which can accommodate the external device and hold it with mechanical or magnetic force.

As shown in FIG. 1, wearable textile body 101 can be a T-shirt. Wearable T-shirt device 100 includes two electrical textile sensors 103a and 103h formed by a plurality of conductive yarns, each of which can function as an electrode. Textile sensors 103a and 103h can be provided at right and left chest areas of the T-shirt, respectively. Textile sensors 103a and 103b can be used to detect, for example, electrocardiogram (ECG) signals of the user's heart. Contacts 105a and 105b respectively connected to textile sensors 103a and 103b can deliver the detected ECG signals to a connected control module.

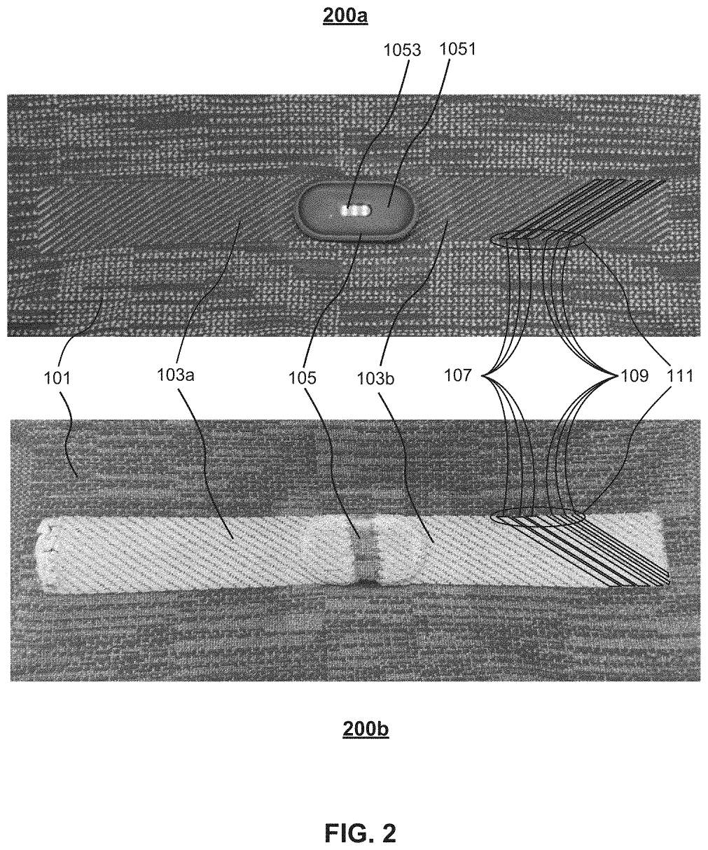

FIG. 2 is an enlarged diagram of textile sensors 103a and 103b of exemplary wearable T-shirt device 100, according to some embodiments of the present disclosure. Enlarged diagram 200a illustrates a front side of textile sensors 103a and 103b of wearable T-shirt device 100, while enlarged diagram 200b illustrates a back side of textile sensors 103a and 103b of wearable T-shirt device 100. The back side of textile sensors 103a and 103b is the inner side which is close to the skin of the user. As shown in enlarged diagrams 200a and 200b of FIG. 2, at areas where textile sensors 103a and 103b are located, functional yarns are integrally interwoven or knitted with nonfunctional yarns of wearable textile body 101. For example, in the area of textile sensor 103b, functional yarns 107 (shown as white yarns) of textile sensor 103b are integrally interwoven or knitted with nonfunctional yarns 109 (shown as gray yarns) of wearable textile body 101 to form a single piece of cloth. At an edge 111 of textile sensor 103b, yarns 109 can be continuous and integrally interwoven or knitted with other nonfunctional yarns to form wearable textile body 101.

As shown in FIG. 2 interface 105 can be provided between textile sensors 103a and 103b and in contact with them at the back side. Interface 105 includes a recess 1051 as a docking station and a plurality of contacts 1053 at the bottom of recess 1051. Recess 1051 can accommodate an external device (not shown in FIG. 2) for electrically conductive connection thereto and hold the external device with mechanical or magnetic force. The external device can include a plurality of corresponding contacts that are electrically connected with contacts 1053 when the external device is accommodated in recess 1051. The external device can communicate with textile sensors 103a and 103h via the contact connection.

Since functional yarns of textile sensors 103a and 103b can be integrally interwoven or knitted with nonfunctional yarns of wearable textile body 101, wearable T-shirt device 100 can improve a wearing experience of the user over conventional wearable devices. In some embodiments, wearable T-shirt device 100 can also provide a high signal to noise rate (SNR) of the detected signals.

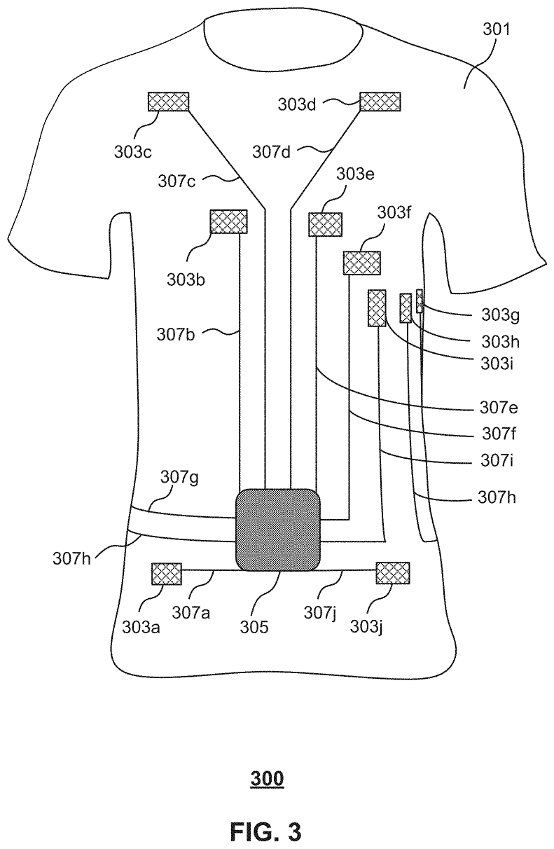

FIG. 3 is a schematic diagram of another exemplary wearable T-shirt device 300, according to some embodiments of the present disclosure. As shown in FIG. 3, wearable T-shirt device 300 includes a wearable textile body 301 and one or more textile sensors, e.g., ten textile sensors 303a-303j, collectively referred to as a textile sensor 303. Although shown as a T-shirt, it is appreciated that wearable textile body 301 can be in the form of other wearable textile items, such as a sweatshirt, underwear, a hat, a wearable band, or the like.

Wearable textile body 301 can be formed of a plurality of nonfunctional yarns which are interwoven or knitted to produce a textile form suitable for wear by a user, such as a human being or other living being. Textile sensor 303 can include a plurality of functional yarns which are integrally interwoven or knitted with the yarns of wearable textile body 301. The functional yarns of textile sensor 303 can be conductive yarn, pressure yarn, strain yarn, or tension yarn, and used to detect various parameters of the user, such as physiological parameters, chemical parameters, local pressure, movement, gesture, or the like. The functional yarn includes a plurality of functional fibers twisted or interlaced together. The functional fiber can include conductive fiber, pressure fiber, strain fiber, tension fiber, or the like. The functional yarn can include a plurality of conductive fibers, pressure fibers, strain fibers, tension fiber, or a combination thereof.

In some embodiments, textile sensor 303 can include a plurality of the same type of functional yarns. For example, textile sensor 303 can be an electrical textile sensor, a pressure textile sensor, a strain textile sensor, or a tension textile sensor. Alternatively, textile sensor 303 can include a plurality of different types of yarns to form a combined textile sensor.

In some embodiments, one or more textile sensors 303 of wearable T-shirt device 300 can be of the same type, such as electrical textile sensor, pressure textile sensor, strain textile sensor, tension textile sensor, combined sensor, or other suitable type of sensors. Alternatively, in some embodiments, one or more textile sensors 303 of wearable T-shirt device 300 can be of different types. For example, wearable T-shirt device 300 can include one or more electrical textile sensors and one or more tension textile sensors.

As shown in FIG. 3, wearable T-shirt device 300 includes textile sensors 303a-303j that are located at different areas of wearable textile body 301. Specifically, textile sensors 303a and 303j are located at right side and left side of the abdominal area of wearable textile body 301, respectively. Textile sensors 303b and 303c are located at the right chest area of wearable textile body 301. Textile sensors 303d-303i are located at the left chest area of wearable textile body 301 and around the heart of the user who wears wearable T-shirt device 300. In some embodiments, each sensor 303 is an electrical textile sensor formed by a plurality of conductive yarns. Textile sensors 303a-303j can be used to detect ECG signals from the user's heart.

In some embodiments, wearable T-shirt device 300 includes an interface 305 that can be coupled to an external device, such as a control module (not shown in FIG. 3), in a wired or wireless manner. For example, interface 305 can include a plurality of contacts for connecting to the external device. As another example, interface 305 can include a wireless transmitter or transceiver which can deliver signals to the external device or receive signals or instructions therefrom. In some embodiments, interface 305 can include a docking station, e.g., a recess, which can accommodate the external device and hold it with mechanical or magnetic force.

As shown in FIG. 3, wearable T-shirt device 300 also includes a plurality of traces, e.g., traces 307a-307j, collectively referred to as a trace 307. Trace 307 can connect textile sensor 303 to interface 305 and deliver signals detected by sensor 303 to interface 305. For example, traces 307a-307j connect textile sensor 303a-303j, respectively, to interface 305. In some embodiments, trace 307 can include a plurality of conductive yarns, the conductive yarn including a plurality of conductive fibers. Conductive yarns of trace 307 can be integrally interwoven or knitted with nonfunctional yarns of wearable textile body 301 and functional yarns of sensor 303. Alternatively, conductive yarns of trace 307 can be attached to wearable textile body 301 and connected to electrical textile sensor 303 formed by a plurality of conductive yarns that is used to detect ECG signals from the user's heart. Trace 307 can deliver the detected ECG signals from sensor 303 to interface 305. Interface 305 can deliver or send the detected ECG signals to the external device, such as a control module, for further processing.

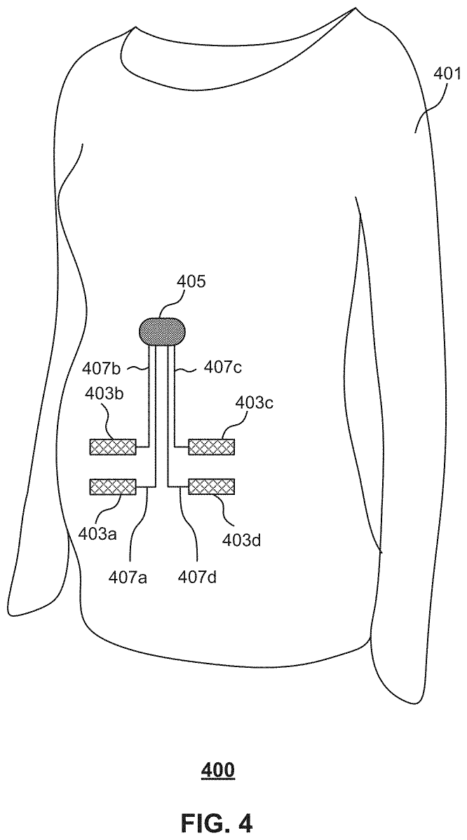

FIG. 4 is a schematic diagram of an exemplary wearable long sleeve T-shirt device 400, according to some embodiments of the present disclosure. Wearable long sleeve T-shirt device 400 can be designed for a pregnant user, such as a pregnant human being or other living being. As shown in FIG. 4, wearable long sleeve T-shirt device 400 includes a wearable textile body 401 and one or more textile sensors, e.g., textile sensors 403a-403d, collectively referred to as a textile sensor 403. Although shown as a long sleeve T-shirt, it is appreciated that wearable textile body 401 can be in the form of other wearable textile items, such as a sweatshirt, underwear, a T-shirt, a wearable band, or the like.

Wearable textile body 401 includes a plurality of nonfunctional yarns which are interwoven or knitted to produce a long sleeve T-shirt suitable for wear by the pregnant user. Textile sensor 403 includes a plurality of functional yarns which are integrally interwoven or knitted with the yarns of wearable textile body 401. The functional yarns of textile sensor 403 can be used to detect various parameters of the user, such as physiological parameters, chemical parameters, local pressure, movement, gesture, or the like. The functional yarn can include a plurality of functional fibers twisted or interlaced together. The functional fiber can include conductive fiber, pressure fiber, strain fiber, tension fiber, or the like. The functional yarn can include a plurality of conductive fibers, pressure fibers, strain fibers, tension fiber, or a combination thereof. Textile sensor 403 can include a plurality of the same type of functional yarns or a plurality of different types of yarns. One or more textile sensors 403 can be of the same type or different types.

As shown in FIG. 4, wearable long sleeve T-shirt device 400 includes textile sensors 403a-403d that are located at different areas over the abdominal area of wearable textile body 401. In some embodiments, each of textile sensors 403a-403d is an electrical textile sensor formed by a plurality of conductive yarns. Textile sensors 403a-403d can be used to detect ECG signals, maternal heart rate (MHR) signals, fetal heart rate (FHR) signals, electromyogram (EMG) activity signals, or the like. EMG activity can include uterine activities, such as uterine contractions (UC). The areas at which textile sensors 403a-403d are located on wearable textile body 401 can be designed to adapt to the changing of position of and the growing of a fetus inside the user's womb. Therefore, wearable long sleeve T-shirt device 400 can monitor in real-time the situations of pregnant user and fetus.

In some embodiments, wearable long sleeve T-shirt device 400 includes an interface 405 that can be coupled to an external device, such as a control module, a smart device, or a computer (not shown in FIG. 4), in a wired or wireless manner. For example, interface 405 can include a plurality of contacts for connecting to the external device. As another example, interface 405 can include a wireless transmitter or transceiver which can transmit the detected signals, e.g., ECG signals, MHR signals, FHR signals, or EMG signals (e.g., UC signals), to the external device or receive signal or instructions therefrom. In some embodiments, interface 305 can include a docking station, e.g., a recess, which can accommodate the external device and hold it with mechanical or magnetic force.

As shown in FIG. 4, wearable long sleeve T-shirt device 400 can also include a plurality of traces, e.g., traces 407a-407d (collectively referred to as a trace 407). Trace 407 can connect textile sensor 403 to interface 405. For example, traces 407a-407d connect textile sensors 403a-403d, respectively, to interface 405. In some embodiments, trace 407 can include a plurality of conductive yarns. The conductive yarns of trace 407 can be integrally interwoven or knitted with nonfunctional yarns of wearable textile body 401 and functional yarns of sensor 403. Alternatively, the conductive yarns of trace 407 can be attached to wearable textile body 401 and connected to sensor 403. Trace 407 can deliver the detected signals, e.g., ECG signals, MHR signals, FHR signals, or UC signals, from sensor 403 to interface 405. Interface 405 can deliver or send the detected signals to the external device, such as a control module, for further processing.

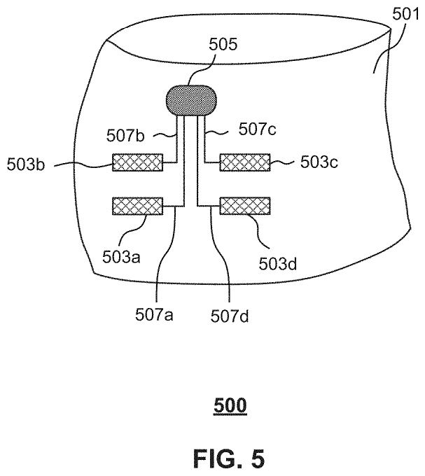

FIG. 5 is a schematic diagram of an exemplary wearable band device 500, according to some embodiments of the present disclosure. As shown in FIG. 5, wearable band device 500 can include a wearable textile body 501 and one or more textile sensors, e.g., 503a-503d, collectively referred to as a textile sensor 503). Although shown as a tubular band, it is appreciated that wearable textile body 501 may have open ends and can be tied on head, body, arm, or leg of a user. Textile sensor 503 can be similarly designed and located as textile sensor 103 of FIG. 1, textile sensor 303 of FIG. 3, or textile sensor 403 of FIG. 4. Similar to wearable long sleeve T-shirt device 400 of FIG. 4, wearable band device 500 can be designed for a pregnant user, such as a pregnant human being or other living being. Wearable band device 500 can be worn around the abdominal area of the pregnant user to perform maternal and fetal monitoring. Wearable textile body 501 can include a plurality of nonfunctional yarns which are interwoven or knitted to produce a band. Textile sensor 503 can include a plurality of functional yarns, e.g., conductive yarns, pressure yarns, strain yarns, tension yarns, or the like, which are integrally interwoven or knitted with the nonfunctional yarns of wearable textile body 501. The functional yarns of textile sensor 503 can include a plurality of functional fibers, e.g., conductive fibers, pressure fibers, strain fibers, tension fibers, or a combination thereof, twisted or interlaced together. Textile sensor 503 can include a plurality of the same type of functional yarns or a plurality of different types of yarns. One or more textile sensors 503 can be of the same type or different types.

In some embodiments, textile sensors 503a-503d each can be an electrical textile sensor formed by a plurality of conductive yarns and function as an electrode. Textile sensors 503a-503d can be used to detect ECG signals, MHR signals, FHR signals, UC signals, or the like. The areas at which textile sensors 503a-503d are located on wearable textile body 501 can be designed to adapt to the changing of position of and the growing of a fetus inside the user's womb.

In some embodiments, wearable band device 500 includes an interface 505 that can be coupled to an external device, such as a control module (not shown in FIG. 5), in a wired or wireless manner. Wearable band device 500 can also include a plurality of traces, e.g., traces 507a-507d, collectively referred to as a trace 507, that connect textile sensors 503a-503d, respectively, to interface 505. Trace 507 can include a plurality of conductive yarns that are integrally interwoven or knitted with nonfunctional yarns of wearable textile body 501 and functional yarns of sensor 503. Alternatively, the conductive yarns of trace 507 can be attached to wearable textile body 501 and connected to sensor 503. Trace 507 can deliver signals, e.g., ECG signals, MHR signals, FHR signals, or UC signals, detected by sensor 503 to interface 505. Interface 505 can deliver or send the detected signals to the external device, such as a control module, for further processing.

In some embodiments, wearable band device 500 can be a head band. A plurality of textile sensors 503 each can be an electrical textile sensor formed by a plurality of conductive rams and function as an electrode. Textile sensors 503 can be used to detect electroencephalogram (EEG) of the user.

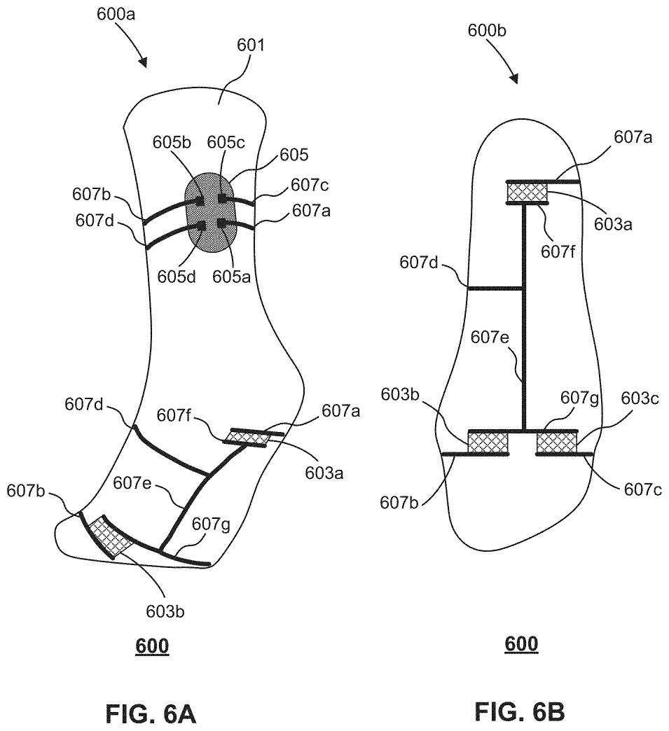

FIGS. 6A and 6B are a schematic diagrams of an exemplary wearable sock device 600, according to some embodiments of the present disclosure. FIG. 6A illustrates a perspective back view 600a of wearable sock device 600, while FIG. 6B illustrates a bottom view 600b of wearable sock device 600. As shown in FIGS. 6A-6B, wearable sock device 600 can include a wearable textile body 601 and one or more textile sensors, e.g., textile sensors 603a-603c, collectively referred to as a textile sensor 603. Although shown as a sock, it is appreciated that wearable textile body 601 can be in the form of other wearable textile items, such as an insole, a shoe, or the like.

Wearable textile body 601 includes a plurality of yarns which are interwoven or knitted to produce a sock suitable for wear by a user, such as a human being or other living being. The plurality of yarns of wearable textile body 601 can be nonfunctional. Textile sensor 603 includes a plurality of functional yarns, e.g., conductive yarns, pressure yarns, strain yarns, tension yarns, or the like, which are integrally interwoven or knitted with of the nonfunctional yarns of wearable textile body 601. The functional yarns of textile sensor 603 can be used to detect various parameters of the user, such as physiological parameters, chemical parameters, local pressure, movement, gesture, or the like. The functional yarn can include a plurality of functional fibers, e.g., conductive fibers, pressure fibers, strain fibers, tension fibers, or a combination thereof, twisted or interlaced together. Textile sensor 603 can include a plurality of the same type of functional yarns or a plurality of different types of yarns. One or more textile sensors 603 can be of the same type or different types.

As shown in FIGS. 6A-6B, for example, wearable sock device 600 includes textile sensors 603a-603c that are located at different areas at the bottom of wearable textile body 601. Specifically, textile sensor 603a is located at the heel of wearable textile body 601. Textile sensors 603b and 603c are located at the left side and right side on the sole of wearable textile body 601. In some embodiments, each textile sensor 603 can include a plurality of pressure yarns to detect a pressure on a foot of the user.

As shown in FIGS. 6A-6B, wearable sock device 600 also includes a plurality of traces, e.g., traces 607a-607g, collectively referred to as a trace 607. In some embodiments, trace 607 can include a plurality of conductive yarns that can be integrally interwoven or knitted with nonfunctional yarns of wearable textile body 601. Alternatively, the conductive yarns of trace 607 can be attached to wearable textile body 601. Traces 607a and 607f are connected to two opposite sides of textile sensor 603a by, for example, being integrally interwoven or knitted with or attached to functional yarns of textile sensor 603a. Similarly, traces 607b and 607g are connected to two opposite sides of textile sensor 603b, and traces 607c and 607g are connected to two opposite sides of textile sensor 603c. When a pressure is applied, e.g., when the user is walking or running, electrical resistances of the pressure fibers of textile sensor 603a, 603b, or 603c change. Traces 607a and 607f, traces 607b and 607g, or traces 607c and 607g can be used to deliver signals indicating the change in electrical resistance of and thus pressure on textile sensor 603a, 603b or 603c, respectively.

In some embodiments, wearable sock device 600 includes an interface 605 that can be coupled to an external device, such as a control module (not shown in FIGS. 6A-6B), in a wired or wireless manner. For example, as shown in FIGS. 6A-6B, interface 605 can include a plurality of contacts 605a-605d for connecting to the external device. Trace 607a, 607b, or 607c can connect one side of textile sensor 603a, 603b, or 603c to contact 605a, 605b, or 605c, respectively. Trace 607d can connect the other side of textile sensors 603a, 603b, or 603c to contact 605d. Specifically, trace 607e connects traces 607f and 607g to trace 607d that connects to contact 605d. Traces 607a-607g can deliver the detected signals from textile sensors 603a-603c to interface 605. Interface 605 can include a docking station, e.g., a recess, which can accommodate the external device and hold it with mechanical or magnetic force. Interface 605 can deliver the detected signals to the external device via contacts 605a-605d. In some embodiments, interface 605 can include a wireless transmitter or transceiver which can transmit signals from textile sensors 603a-603c to the external device.

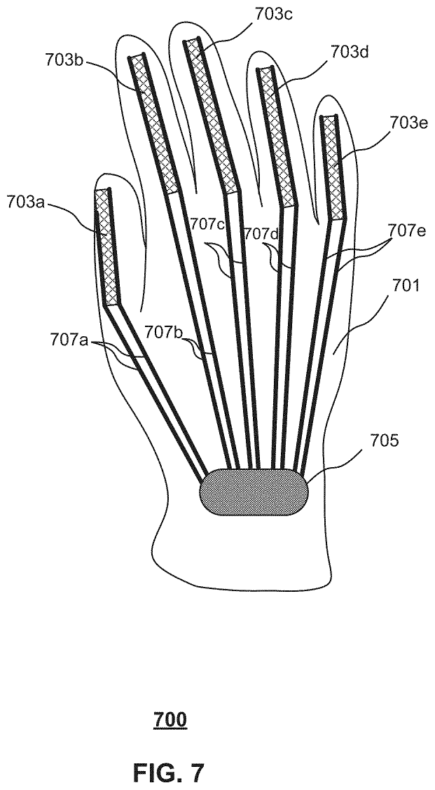

FIG. 7 is a schematic diagram of an exemplary wearable glove device 700, according to some embodiments of the present disclosure. As shown in FIG. 7, wearable glove device 700 includes a wearable textile body 701 and one or more textile sensors, e.g., textile sensors 703a-703e, collectively referred to as a textile sensor 703. Although shown as a glove, it is appreciated that wearable textile body 701 can be in the form of other wearable textile items, such as a sweatshirt, a T-shirt, underwear, a wearable band, or the like.

Wearable textile body 701 can be formed of a plurality of nonfunctional yarns which are interwoven or knitted to produce a glove suitable for wear by a user, such as a human being or other living being. Textile sensor 703 can include a plurality of functional yarns, e.g., conductive yarns, pressure yarns, strain yarns, tension yarns, or the like, which are integrally interwoven or knitted with the yarns of wearable textile body 701. The functional yarns of textile sensor 703 can be used to detect various parameters of the user, such as physiological parameters, chemical parameters, local pressure, movement, gesture, or the like. The functional yarn can include a plurality of functional fibers, e.g., conductive fibers, pressure fibers, strain fibers, tension fibers, or a combination thereof, twisted or interlaced together. Textile sensor 703 can include a plurality of the same type of functional yarns or a plurality of different types of yarns. One or more textile sensors 703 can be of the same type or different types.

As shown in FIG. 7, for example, wearable glove device 700 includes textile sensors 703a-703e that are located at back sides of five fingers of wearable textile body 701. Specifically, textile sensors 703a-703e are located at and extend along the back sides of the thumb, forefinger, middle finger, third finger, and little finger of wearable textile body 701, respectively. In some embodiments, each textile sensor 703 includes a plurality of tension yarns that can be used to detect a tension along a corresponding finger and trace a gesture or movement of the finger.

As shown in FIG. 7, wearable glove device 700 also includes multiple pairs of traces, e.g., trace pairs 707a-707e, collectively referred to as a trace pair 707. In some embodiments, each trace in trace pair 707 can include a plurality of conductive yarns that can be integrally interwoven or knitted with nonfunctional yarns of wearable textile body 701. Alternatively, conductive yarns of trace pair 707 can be attached to wearable textile body 701. Trace pairs 707a, 707b, 707c, 707d, and 707e are connected to two opposite sides of textile sensor 703a, 703b, 703c, 703d, and 703e, respectively, by, for example, being integrally interwoven or knitted with or attached to functional yarns of the corresponding textile sensor. When a user changes the gesture of their fingers, e.g., bending fingers, one or more of textile sensors 703a-703e can be stretched or released. The electrical resistances of the tension yarns, e.g., tension fibers, of textile sensor 703a, 703b, 703c, 703d, or 703e may change as the tensions in the tension yarns change. Trace pair 707a, 707b, 707c, 707d, or 707e can deliver signals indicating a change in electrical resistance of, and thus tension on, textile sensor 703a, 703b, 703c, 703d, or 703e, respectively.

In some embodiments, wearable glove device 700 can include an interface 705 that can be coupled to an external device, such as a control module (not shown in FIG. 7), in a wired or wireless manner. For example, interface 705 can include a plurality of contacts for connecting to the external device. Interface 705 can include a docking station, e.g., a recess, which can accommodate the external device and hold it with mechanical or magnetic force. Trace pairs 707a, 707b, 707c, 707d, and 707e can connect textile sensors 703a, 703b, 703c, 703d, and 703e, respectively, to interface 705. Interface 705 can deliver the detected signals from textile sensor 703 to the external device via contacts. In some embodiments, interface 705 can include a wireless transmitter or transceiver which can transmit signals from textile sensor 703 to the external device.

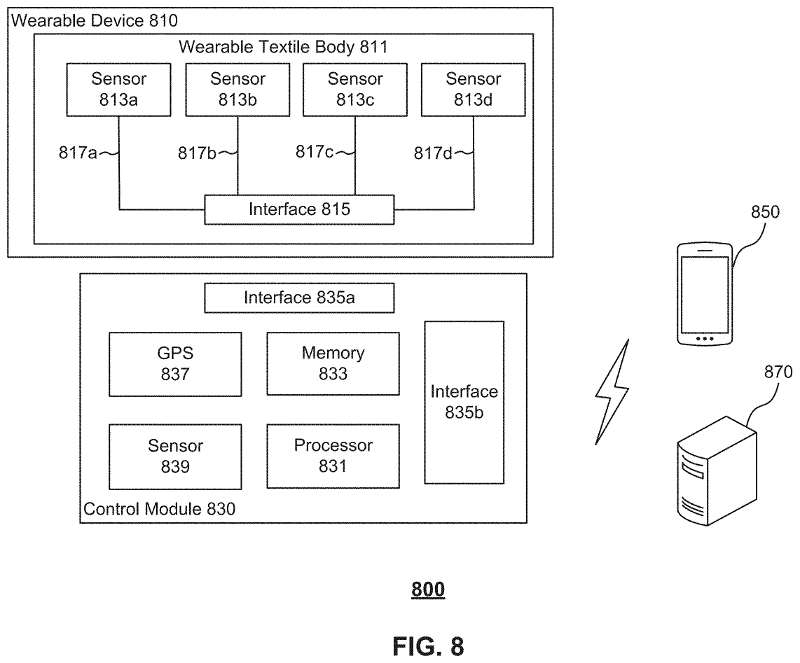

FIG. 8 is a schematic diagram of an exemplary system 800 including a wearable device 810, according to some embodiments of the present disclosure. As shown in FIG. 8, system 800 includes a wearable device 810 and a control module 830. It is appreciated that wearable device 810 can be wearable T-shirt device 100 of FIGS. 1-2, wearable T-shirt device 300 of FIG. 3, wearable long sleeve T-shirt device 400 of FIG. 4, wearable band device 500 of FIG. 5, wearable sock device 600 of FIGS. 6A-6B, or wearable glove device 700 of FIG. 7. Wearable device 810 includes a wearable textile body 811 and one or more textile sensors, e.g., textile sensors 813a-813d, collectively referred to as a textile sensor 813. It is appreciated that wearable textile body 811 can have the form of any suitable wearable textile items, such as a sweatshirt, underwear, T-shirt, a long sleeve T-shirt, a sock, a glove, a hat, a wearable band, or the like.

Wearable textile body 811 includes a plurality of yarns which are interwoven or knitted to produce a textile form suitable for wear by a user, such as a human being or other living being. Wearable textile body 811 can be formed of a plurality of nonfunctional yarns. Textile sensor 813 can include a plurality of functional yarns which are integrally interwoven or knitted with a part of the plurality of yarns of wearable textile body 811. The functional yarns, e.g., conductive yarns, pressure yarns, strain yarns, tension yarns, or the like, of textile sensor 813 can be used to detect various parameters of the user, such as physiological parameters, chemical parameters, local pressure, movement, gesture, or the like. The functional yarn can include a plurality of functional fibers, e.g., conductive fibers, pressure fibers, strain fibers, tension fibers, or a combination thereof, twisted or interlaced together.

In some embodiments, textile sensor 813 can include a plurality of the same type of functional yarns. For example, textile sensor 813 can include a plurality of conductive yarns, pressure yarns, strain yarns, or tension yarns to form an electrical textile sensor, a pressure textile sensor, a strain textile sensor, or a tension textile sensor, respectively. Alternatively, textile sensor 813 can include a plurality of different types of yarns to form a combined textile sensor. For example, textile sensor 813 can include a plurality of conductive yarns and pressure yarns to act as a combination of tension textile sensor and pressure textile sensor.

In some embodiments, one or more textile sensors 813 can be of the same type, such as electrical textile sensor, pressure textile sensor, strain textile sensor, tension textile sensor, combined sensor, or other suitable type of sensors. Alternatively, in some embodiments, one or more textile sensors 813 can be of different types. For example, wearable device 810 can include one or more electrical textile sensors and one or more tension textile sensors.

In some embodiments, wearable device 810 includes an interface 815 that can be connected with textile sensor 813 directly or indirectly. For example, as shown in FIG. 8, wearable device 810 can also include a plurality of traces or trace pairs, e.g., traces 817a-817d, collectively referred to as a trace 817. Traces 817a-817d connect textile sensors 813a-813d, respectively, to interface 815 and deliver signals detected by textile sensors 813a-813d to interface 815. In some embodiments, each trace 817 can include a plurality of conductive yarns that are integrally interwoven or knitted with nonfunctional yarns of wearable textile body 811. Alternatively, the conductive yarns of trace 817 can be attached to wearable textile body 811. Traces 817a-817d are connected to textile sensors 813a-813d, respectively, by, for example, being integrally interwoven or knitted with or attached to functional yarns of the textile sensors.

As shown in FIG. 8, control module 830 includes a processor 831, a memory 833, and an interface 835a. Interface 835a can be wiredly or wirelessly coupled to interface 815 of wearable device 810. For example, interface 835a can include a plurality of contacts for connecting to corresponding contacts on interface 815. As another example, interface 835a can include a wireless transmitter, receiver, or transceiver to communicate with interface 815. In some embodiments, interface 815 can include a docking station, e.g., a recess, which can accommodate the control module 830 and hold it with mechanical or magnetic force.

Memory 833 can store data, signals, or instructions. For example, memory 833 can store signals received from wearable device 810 through interface 835a. Memory 833 can also store data for processor 831 and instructions performed by processor 831. Memory 833 can be of any suitable form, including, but is not limited to, removable or nonremovable, volatile or non-volatile Read Only Memory (ROM), Random Access Memory (RAM), flash, or the like.

Processor 831 can process or pre-process signals received from wearable device 810. The received signals include, but are not limited to, ECG signals, MI-IR signals, FHR signals, EMG signals, e.g., UC signals, EEG signals, pressure signals, tension signals, strain signals or the like. For example, processor 831 can pre-process received signals to filter noise. Processor 831 can use a model or an algorithm to identify effective signals from received signals, such as heart beat signals. Processor 831 can transform received signals to data or signals indicating a situation, e.g., health, movement, gesture, or the like, of the user. In some embodiments, processor 831 can also encrypt data that are to be transmitted outside to protect private information of the user.

In some embodiments, control module 830 can include a GPS unit 837 and one or more sensors 839. GPS unit 837 can provide location information of the user if control module 830 is held in interface 815 of wearable device 810. One or more sensors 839 can include, but are not limited to, a gravity sensor, an accelerometer, a magnetic field sensor, a gyroscope, or the like, to provide additional data of system 800.

In some embodiments, control module 830 can include another interface 835b that can communicate with external devices, such as a smart terminal 850 or a server 870, in a wired or wireless manner. For example, interface 835b can communicate with external devices via Universal Serial Bus (USB) or Bluetooth. Smart terminal 850 can include, but is not limited to, a cellular phone, a laptop, a tablet computer, a Personal Digital Assistant (PDA), or the like. Smart terminal 850 or server 870 can further process data from control module 830. In some embodiments, smart terminal 850 and server 870 can transmit received or further processed data to a cloud or a hospital.

Embodiments of the present disclosure provides improvements over conventional wearable devices. For example, in some embodiments, functional yarns of a textile sensor can be integrally interwoven or knitted with nonfunctional yarns of a wearable textile body. This can provide a more comfortable wearing experience to the user. In some embodiments, the comfortable wearing experience can be further enhanced by integrally interweaving or knitting conductive yarns of trace with nonfunctional yarns of wearable textile body. In addition, the wearable device according to some embodiments of the present disclosure can provide real-time monitoring with high SNR for the user.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as nonexclusive. Further, the steps of disclosed methods can be modified in any manner, including reordering steps and/or inserting or deleting steps.

The features and advantages of the present disclosure are apparent from the detailed specification, and thus, it is intended that the appended claims cover all systems and methods falling within the true spirit and scope of the present disclosure. As used herein, the indefinite articles “a” and “an” mean “one or more.” Further, since numerous modifications and variations will readily occur from studying the present disclosure, it is not desired to limit the present disclosure to the exact reconstruction and operation illustrated and described, and accordingly, all suitable modifications and equivalents may be resorted to, falling within the scope of the present disclosure.

As used herein, unless specifically stated otherwise, the term “or” encompasses all possible combinations, except where infeasible. For example, if it is stated that a component may include A or B, then, unless specifically stated otherwise or infeasible, the component may include A, or B, or A and B. As a second example, if it is stated that a component may include A, B, or C, then, unless specifically stated otherwise or infeasible, the component may include A, or B, or C, or A and B, or A and C, or B and C, or A and B and C.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A wearable device, comprising:
a wearable textile body comprising a plurality of interwoven or knitted yarns;
one or more textile sensors each located at an area on the wearable textile body, each textile sensor comprising a plurality of functional yarns integrally interwoven or knitted with the yarns of the wearable textile body at the located area; and
an interface communicatively coupled with the one or more textile sensors.

2. The wearable device of claim 1, wherein the functional yarns of at least one of the one or more textile sensors comprise conductive yarns, and the interface is in electrically conductive contact with the at least one of the one or more textile sensors.

3. The wearable device of claim 1, wherein the one or more textile sensors comprise two textile sensors each comprising a plurality of conductive yarns, and the interface is in electrically conductive contact with the two textile sensors.

4. The wearable device of claim 1, further comprising:
one or more traces connecting the one or more textile sensors to the interface.

5. The wearable device of claim 4, wherein each of the traces comprises a plurality of conductive yarns integrally interwoven or knitted with or attached to the yarns of the wearable textile body.

6. The wearable device of claim 4, wherein the functional yarns of at least one of the one or more textile sensors comprise conductive yarns, and the one or more traces comprise at least one trace having a plurality of conductive yarns integrally interwoven or knitted with or attached to the conductive yarns of the at least one of the one or more textile sensors.

7. The wearable device of claim 4, wherein the one or more textile sensors comprise ten textile sensors each comprising a plurality of conductive yarns, and the one or more traces comprise ten traces each having a plurality of conductive yarns integrally interwoven or knitted with or attached to the conductive yarns of one textile sensor of the ten textile sensors and connecting the one textile sensor to the interface.

8. The wearable device of claim 1, wherein the interface comprises a docking station and a plurality of contacts electrically connected to the textile sensors.

9. The wearable device of claim 1, wherein the plurality of functional yarns of the textile sensor comprises conductive yarns, pressure yarns, strain yarns, tension yarns, or a combination of two or more different functional yarns.

10. The wearable device of claim 1, wherein the functional yarns of the textile sensor each comprises a plurality of conductive fibers, pressure fibers, strain fibers, tension fibers, or a combination of fibers of two or more different functional yarns, twisted or interlaced together.

11. The wearable device of claim 1, wherein the wearable textile body is in a form of a sweatshirt, a T-shirt, a long sleeve T-shirt, underwear, a hat, or a wearable band.

12. The wearable device of claim 1, wherein the one or more textile sensors are configured to detect one or more of electrocardiogram (ECG) signals, maternal heart rate (MHR) signals, fetal heart rate (FHR) signals, electromyogram (EMG) signals, Electroencephalogram (EEG) signals, pressure signals, tension signals, or strain signals.

13. A system, comprising:
a wearable device comprising:
a wearable textile body comprising a plurality of interwoven or knitted yarns;
one or more textile sensors each located at an area on the wearable textile body, each textile sensor comprising a plurality of functional yarns integrally interwoven or knitted with the yarns of the wearable textile body at the located area; and
an interface communicatively coupled with the one or more textile sensors; and
a control module, comprising:
an interface to receive signals from the wearable device;
a memory for storing the received signals or data; and
a processor coupled with the memory and to process the received signals.

14. The system of claim 13, wherein the functional yarns of at least one of the one or more textile sensors comprise conductive yarns, and the interface of the wearable device is in electrically conductive contact with the at least one of the one or more textile sensors.

15. The system of claim 13, wherein the one or more textile sensors comprise two textile sensors each comprising a plurality of conductive yarns, and the interface is in electrically conductive contact with the two textile sensors.

16. The system of claim 13, wherein the wearable device further comprises:
one or more traces connecting the one or more textile sensors to the interface, each trace comprising a plurality of conductive yarns integrally interwoven or knitted with or attached to the yarns of the wearable textile body.

17. The system of claim 16, wherein the functional yarns of at least one of the one or more textile sensors comprise conductive yarns, and the one or more traces comprise at least one trace having a plurality of conductive yarns integrally interwoven or knitted with or attached to the conductive yarns of the at least one of the one or more textile sensors.

18. The system of claim 16, wherein the one or more textile sensors comprise ten textile sensors each comprising a plurality of conductive yarns, and the one or more traces comprise ten traces each having a plurality of conductive yarns integrally interwoven or knitted with or attached to the conductive yarns of one textile sensor of the ten textile sensors and connecting the one textile sensor to the interface.

19. The system of claim 13, wherein the interface of the wearable device comprises a docking station for accommodating the control module and a plurality of contacts for connection with a plurality of corresponding contacts of the interface of the control module.

20. The system of claim 13, wherein the interface of the control module is a first interface, and the control module further comprising:
a GPS unit;
one or more sensors; and
a second interface to communicate with a smart device or a server.
